(19) United States
(12) Patent Application Publication
(10) Pub. No.: US 2011/0253144 A1
(43) Pub. Date: Oct. 20, 2011

(54) Nasal Interface for Continuous Positive Airway Pressure Mask

(76) Inventor: David Groll, Murrysville, PA (US)

(21) Appl. No.: 12/905,404

(22) Filed: Oct. 15, 2010

Related U.S. Application Data
(63) Continuation-in-part of application No. 12/070,463, filed on Feb. 19, 2008.

(51) Int. Cl. A61M 16/06

(52) U.S. Cl. 128/206.24

(57) ABSTRACT

The mask that is the subject of the present invention enables the delivery of continuous positive airway pressure to patients suffering from sleep apnea by engaging and sealing the area surrounding the patient's nasal passages in order to deliver the therapy. The present invention includes a new nasal interface piece that is of different material composition than the balance of the mask and is comprised of between twelve and thirty-two percent elastane, between twelve and thirty-two percent polyurethane and between forty-six and sixty-six percent polyamide.

### CROSS-REFERENCE TO RELATED APPLICATION

This patent application is a continuation-in-part and claims priority from pending U.S. application Ser. No. 12/070,463, filed Feb. 19, 2008.

### FIELD OF THE INVENTION

The present invention relates, in general, to equipment used in the treatment of sleep apnea and other respiratory ailments and, more particularly, the instant invention relates to an improved nasal interface for an apparatus to deliver a fluid pressure to a patient in order to maintain the patient's airway open while sleeping and deliver oxygen to such patient.

### BACKGROUND OF THE INVENTION

As is well known in the medical field, sleep apnea is a disorder that affects more than 12 million people in the United States alone. It takes its name from the Greek word apnea, which means “without breath.” People with sleep apnea literally stop breathing repeatedly during their sleep, often for a minute or longer, and as many as hundreds of times during a single night.

Sleep apnea is known to be caused by either complete obstruction of the airway (obstructive apnea) or partial obstruction (obstructive hypopnea), both of which can cause the person suffering from such sleep apnea to wake up, temporarily, in order to breathe. There are three types of sleep apnea—obstructive, central, and mixed. Of these, obstructive sleep apnea (OSA) is the most common. OSA occurs in approximately 2 percent of women and 4 percent of men over the age of 35.

The exact cause of OSA remains unclear. The site of obstruction in most patients is the soft palate, extending to the region at the base of the tongue. There are no rigid structures, such as cartilage or bone, in this area to hold the airway open. During the day, muscles in the region keep the passage wide open. But as a person with OSA falls asleep, these muscles relax to a point where the airway collapses and breathing becomes impossible. When breathing stops, the sleeper partially awakens, involuntarily tenses the muscles in the region to open the airway in order to breathe, and falls back asleep. The arousal from sleep usually lasts only a few seconds, but these brief arousals disrupt continuous sleep and prevent the person from reaching the deep stages of slumber, such as rapid eye movement (REM) sleep, which the body needs in order to rest and replenish its strength. Even though normal breathing is restored when the person awakens briefly, the cycle is repeated throughout the night. Typically, the frequency of waking episodes is somewhere between 10 and 60, although a person with severe OSA may have more than 100 waking episodes in a single night.

Positive airway pressure has been demonstrated to be a very effective treatment for obstructive sleep apnea. It has three forms: continuous positive airway pressure (CPAP), autotitration, and bi-level positive airway pressure (BIPAP). While positive airway pressure is usually easier to tolerate at lower pressures, every patient requires a different pressure. In order to determine each individual patient's optimum airway pressure, it is necessary to titrate the pressure to each individual patient during a polysomnogram. A polysomnogram will show not only when the respiratory events have ceased, but also when the arousals from the respiratory events occur.

CPAP, the most common of the three therapy modes, is usually administered at bedtime through a facial mask held in place by straps around the patient's head. The mask is connected by a tube to a small air compressor about the size of a shoe box. The CPAP machine sends air under pressure through the tube into the mask, where, assuming a good seal with the patient's face, it applies a positive air pressure to the upper airways. This positive air pressure essentially “splints” the upper airway open and keeps it from collapsing.

Approximately 55 percent of patients who use CPAP do so on a nightly basis for more than four hours. The advantages of CPAP are that it is very safe and completely reversible. Generally, the treatment is well tolerated; however, it suffers from the disadvantage that it requires active participation every night; that is, the patient must put it on for it to work. If the mask is ill-fitting or causes unwanted side effects, compliance will not be maintained.

Nasal CPAP Masks on the market today fall into one of three design categories. They are either nasal masks, full face masks (i.e., those which cover the mouth and nose) or nasal pillows. The vast majority of CPAP masks that are currently on the market are comprised of a rigid plastic frame buffered by an elastomeric “cushion” that makes the actual contact with the patient's face. These masks are sealed to the patient's face by tightening straps that hold the mask to the patient's head to increase mechanical pressure against and into the skin of the face. The skin and tissue of the patient's face is compressed by the mask, which forms a “gasket” that creates a seal and keeps the air pressure inside the mask elevated. Unfortunately, aside from being uncomfortable, a disadvantage to this approach is that the mechanical pressure applied to the patient's skin often exceeds the perfusion pressure in the tissue under the skin. Consequently blood flow to the tissue is diminished or cut off entirely. This leads to pain and can ultimately cause pressure sores on the patient's face.

These problems were largely overcome by the soft cloth mask disclosed in U.S. patent application Ser. No. 12/070,463, (the “'463 application”). The '463 application disclosed a nasal CPAP mask that was entirely comprised of a non-rigid, compliant material that had no fixed shaped unless breathable gas at positive pressure was flowing through it. While the nasal mask of the '463 application remedied most of the drawbacks of the prior art, it still suffered from air leakage at the interface between the patient's nose and the mask, due largely to the manner in which the nasal interface was constructed. The air leakage was a problem for many reasons, including, reduction of positive pressure applied to the patient's airway, dry eyes from air blowing into them, disruption of sleep of bed partner caused by both blowing jets of air and noise associated therewith.

The present invention resolves the air leakage problem and is an improvement upon the nasal interface of the mask of the '463 application. Furthermore, it is even reported to be more comfortable than the prior art mask, thus leading to increased compliance rates and better patient outcomes.

### SUMMARY OF THE INVENTION

It is, therefore, one of the primary objects of the present invention to provide an improved nasal interface for an apparatus for communicating a positive fluid pressure to a patient's nasal and/or air passageways which has improved air sealing capability while still being more comfortable for the patient.

A further object of the present invention is to provide an apparatus for communicating a positive fluid pressure to a patient's nasal and/or air passageways wherein substantially all portions of such apparatus that touch a patient's face are made of a stretchy composite material that is substantially capable of conforming to the patient's facial features, and in which there are no rigid parts to press against the patient's skin

In addition to the various objects and advantages of the present invention which have been described above, various other objects and advantages of the invention will become more readily apparent to those persons skilled in the relevant art from the following more detailed description of the invention, particularly, when such description is taken in conjunction with the attached drawing figures and with the appended claims.

### BRIEF DESCRIPTION OF THE DRAWINGS

FIG. 1 is a perspective view of a cloth nasal CPAP mask having a prior art nasal interface.

FIG. 2 is a perspective view of a cloth nasal CPAP mask with a nasal interface according to a presently preferred embodiment of the invention.

FIG. 3 is an elevational view of a cloth nasal CPAP mask with a nasal interface according to a presently preferred embodiment of the invention.

FIG. 4 is an elevational view of a cloth nasal CPAP mask having a prior art nasal interface.

FIG. 5 is a plan view of a cloth pattern used for a prior art cloth nasal CPAP mask.

FIG. 6 is a plan view of two cloth patterns used to create the mask and nasal interface of the present invention.

FIG. 7 is a plan view of a partially assembled mask with the nasal interface piece of the present invention.

### DETAILED DESCRIPTION OF THE INVENTION

Prior to proceeding to the more detailed description of the instant invention it should be noted that identical components having identical functions have been designated with identical reference numerals throughout the several views illustrated in the drawings for the sake of clarity.

As used in the present specification and claims the term “nasal interface” means that portion of the mask that actually makes contact with the patient's nose and face when the CPAP mask is being used as intended.

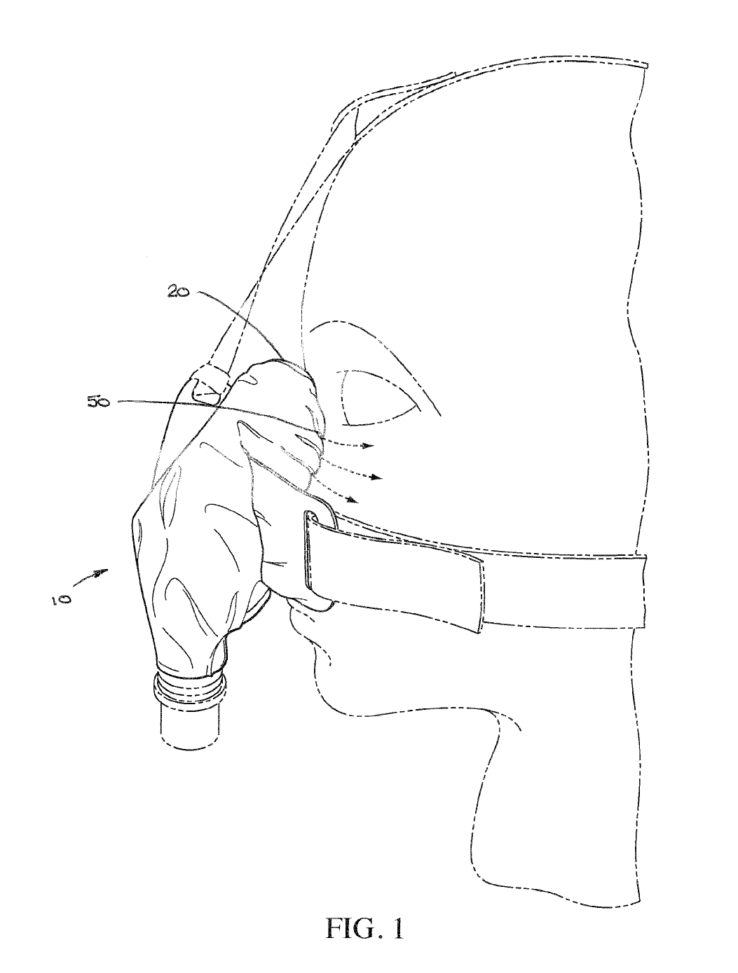

Referring now to FIGS. 1, 4 and 5, a prior art cloth mask 10 is illustrated as being worn by a patient. Generally, the prior art cloth mask 10 has a first open end 20 that forms the nose-receiving space 40 of the previously designed prior art mask 10. In this prior design, the first open end 20 and mask 10 were cut from the same piece of material and were comprised of an inelastic, but compliant, material selected from the group consisting of cloth, relatively soft plastic, rubber, treated paper and various combinations thereof. Alternate embodiments comprised laminated materials with synthetic or natural fibers, woven or non-woven materials, and/or laminates of cloth and/or plastic.

Due to the fact that the material was inelastic, the material in the area of the nasal interface had to be notched and sewn to create a form that would sealingly fit a patient's nose. The result of this notching and sewing was that seams 50 were created. During therapy, then, when the mask 10 was being used to deliver a continuous positive airway pressure to the patient, air leaks (represented by the unnumbered arrows) occurred along the seams 50, leading to undesirable side effects.

Referring specifically to FIG. 4, the prior art mask 10 also had a cushion 80 that was sewn into the mask material to pad the mask 10 in the area of the underside of the patient's nose between the nostrils, called the septum. Additionally, two different sections of the mask material were also folded in this area to reduce irritation to the septum, but some patients still found this area of the mask to create a sore spot after repetitive use required by the therapy.

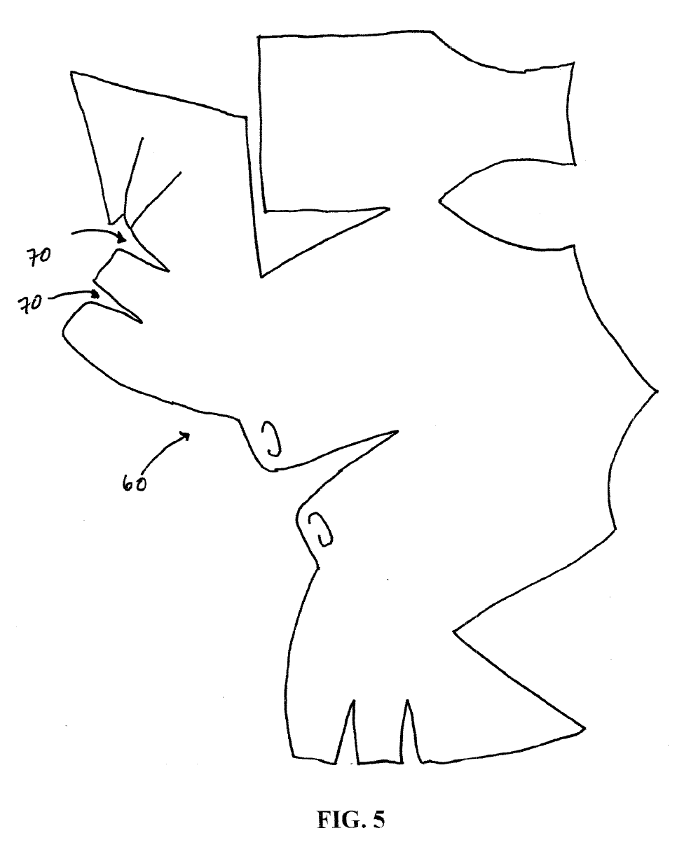

FIG. 5 illustrates the pattern 60 that was cut from a single sheet of material to create the prior art mask 10. When the prior art mask 10 was assembled, the notches 70 in the pattern 60 were sewn together and became the seams 50 (see FIG. 1), along which air previously escaped.

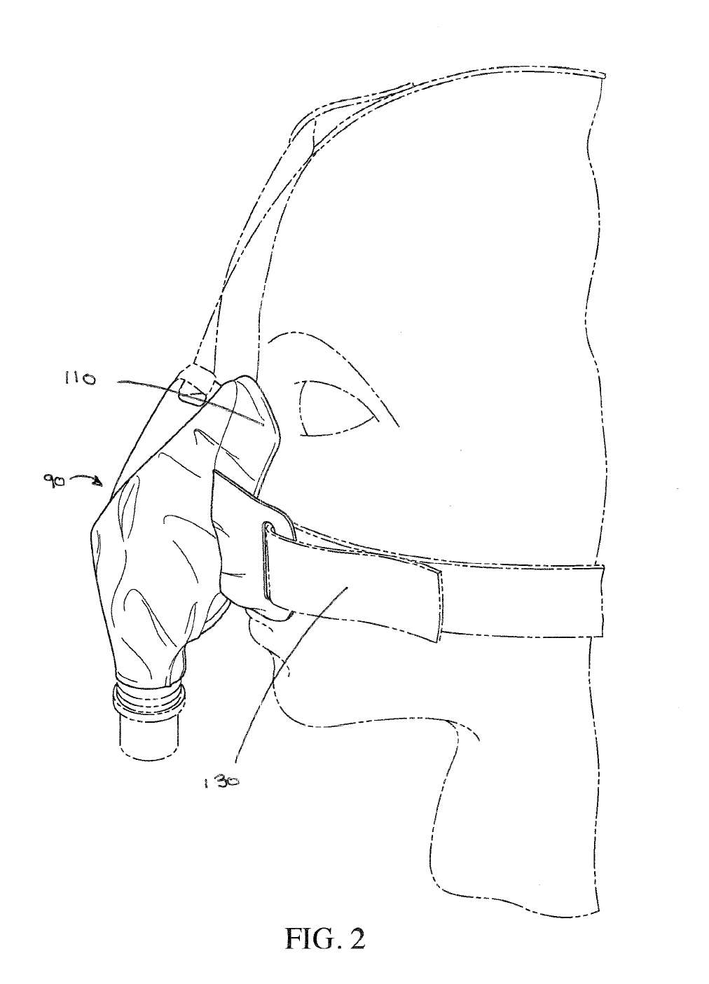

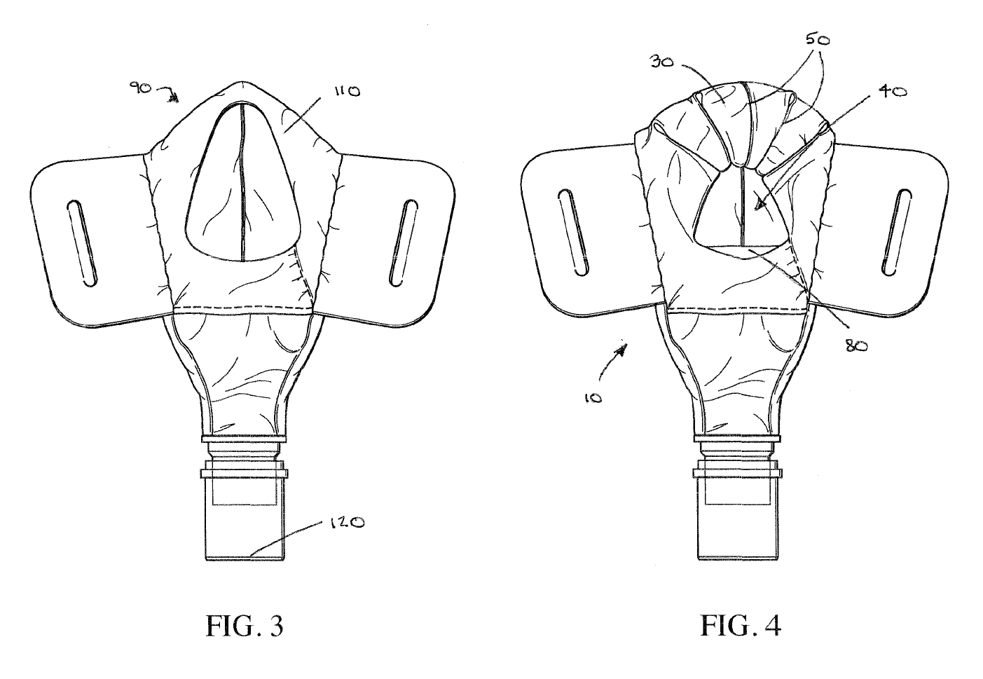

FIGS. 2, 3 and 6 illustrate a preferred embodiment of the mask 90 of the present invention. While the main body 100 of the mask is the same inelastic yet compliant material used in the prior art masks, the nasal interface piece 110 preferably consists of a cloth-like material containing elastane, polyurethane and polyamide, thereby making it both compliant and elastic. For lack of a better term, the nasal interface piece 110 is stretchy. A preferred embodiment is for the nasal interface piece 110 to consist of a material comprising twelve to thirty-two (preferably twenty-two) percent elastane, twelve to thirty-two (preferably twenty-two) percent polyurethane, and forty-six to sixty-six (preferably fifty-six) percent polyamide.

When worn by a patient, the elastic and stretchy quality of the nasal interface piece 110 will cause it to naturally “grab” the patient's nose, but when the CPAP machine is turned on, the air pressure in the mask itself will inflate the mask 90 and, in conjunction with the retaining means 130, thereby cause the nasal interface piece 110 to gently and evenly push against the patient's skin to create the necessary seal.

The cushion that caused irritation to the patient's septum was removed in this new design. In the present invention, the septum is contacted only by the material of the elastic nasal interface 110, which is very thin and comfortable.

As is well known, a fluid communications means may be engaged at the second open end 120 of the mask for communicating positive air pressure. The apparatus also includes a retaining means 130, disposed on the mask 90, for retaining the mask in position on such patient's face during use. Further, the apparatus includes an exhalation valve (not shown) for expiration of exhaled gases.

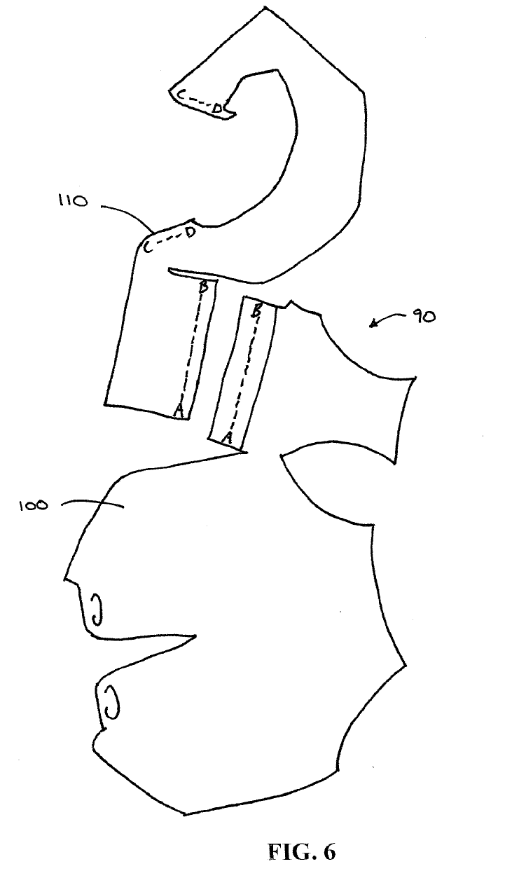

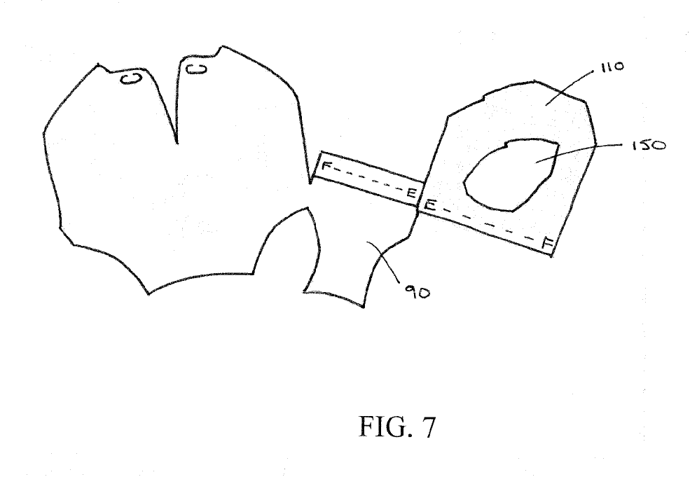

Referring more particularly to FIGS. 6 and 7, assembly of the mask 90 with the nasal interface piece 110 of the present invention will be described. Initially, the nasal interface piece 110 is sewn onto the main body 100 at segment A-B. Then, the nasal interface piece 110 is sewn to itself at segment C-D. FIG. 7 illustrates assembly of the mask 90 with the nasal interface piece 110 sewn in a manner that creates a nose-receiving space 150. The next step in the assembly is to fold segment E-F of the nasal interface piece 110 onto the segment E-F on the main body 90 and sew or otherwise permanently fasten them together. This completes the assembly of the nasal interface piece of the present invention.

If not otherwise stated herein, it is to be assumed that all patents, patent applications, patent publications and other publications (including web-based publications) mentioned and cited herein are hereby fully incorporated by reference herein as if set forth in their entirety herein.

While in accordance with the patent statutes the presently preferred and various alternative embodiments of the instant invention have been described in detail above, it should be understood that various other modifications and alternatives can be envisioned by those persons skilled in the art without departing from either the spirit of the invention or the scope of the appended claims.

What is claimed is:

1. A mask for delivering a breathable gas at positive pressure to a patient's nasal passageways, comprising:
a first open end having a nasal interface piece disposed thereon to form a seal with the patient's nose and adjacent facial area; the nasal interface piece being comprised of a stretchy material;
a second open end having a fluid communication arrangement disposed thereon to securely receive the breathable gas into the mask;
a retaining means for securing the mask to the patient's nose and adjacent facial area.

2. The mask of claim 2, wherein the nasal interface piece is shaped to create a nose-receiving space.

3. The mask of claim 1, wherein the nasal interface piece can be stretched to accommodate a plurality of different nose shapes.

4. The mask of claim 1, wherein the nasal interface piece is substantially seamless.

5. The mask of claim 1, wherein the nasal interface piece has one seam.

6. The mask of claim 1, wherein the stretchy material is comprised of a combination of elastane, polyurethane and polyamide.

7. The mask of claim 6, wherein the percentage of elastane in the stretchy material is in the range of between twelve and thirty-two percent.

8. The mask of claim 6, wherein the percentage of polyurethane in the stretchy material is in the range of between twelve and thirty-two percent.

9. The mask of claim 6, wherein the percentage of polyamide in the stretchy material is in the range of between forty-six and sixty-six percent.

10. An apparatus for use in supplying a breathable gas under continuous positive air pressure to a patient, wherein said apparatus comprises:
a mask having a first open end and a second open end, the first open end being shaped to form a nasal interface piece comprised of a substantially seamless stretchy material formed to create a nose-receiving space;
a fluid communication means disposed on the second end of the mask to securely receive the breathable gas into the mask;
an exhalation valve means disposed on the mask between the first open end and the second open end; and
a retaining means for affixing the mask to the patient's head.

11. The apparatus of claim 10, wherein the substantially seamless stretchy material is comprised of a combination of twenty-two percent elastane, twenty-two percent polyurethane and fifty-six percent polyamide.

12. An apparatus for use in supplying a breathable gas under continuous positive air pressure to a patient wherein the mask has a first open end having a nasal interface piece disposed thereon to form a seal with the patient's nose and adjacent facial area, a second open end having a fluid communication means disposed thereon to securely receive the breathable gas into the mask and a retaining means for securing the mask to the patient's nose and adjacent facial area, wherein the improvement comprises the nasal interface piece being comprised of a stretchy material.

13. A template for creating a nasal interface piece as shown in FIG. 6.
